(19) United States
(12) Patent Application Publication
(10) Pub. No.: US 2011/0071436 A1
(43) Pub. Date: Mar. 24, 2011

(54) AIR CUSHION SENSOR FOR TACTILE SENSING DURING MINIMALLY INVASIVE SURGERY

(76) Inventors: Kaspar Althoefer, London (GB); Dinusha Zbyszewski, London (GB); Pinyo Puangmali, London (GB); Panagiotis Polygerinos, London (GB); Lakmal Seneviratne, London (GB)

(21) Appl. No.: 12/736,957

(22) PCT Filed: Jun. 4, 2009
(86) PCT No.: PCT/GB2009/001403

§ 371 Date: Nov. 24, 2010

(30) Foreign Application Priority Data
Jun. 5, 2008 (GB) 0810317.8

(51) Int. Cl. A61B 5/103; G01N 3/42

(52) U.S. Cl. 600/587; 73/82

(57) ABSTRACT

A sensor is described for detecting the force applied to or by soft material, and that is thus able to measure the stiffness of a soft material. The sensor comprises a sensor body into which is supplied a fluid under pressure. At least one sensor members are provided that are arranged to project from the sensor body under the pressure exerted thereon by the fluid. A sensor member displacement detection system is also provided, that is preferably optically based using optical fibres to illuminate the sensor member and that measures the modulation of the light reflected from the sensor member as the member is displaced against the pressure of the fluid to detect and measure the displacement. From the measured displacement an estimate of the force being applied to the sensor member can be obtained. The sensor is of a small size suitable for use during MIS or catheterisation procedures. Preferably the sensor is constructed of non-metallic material such that it is MRI compatible.

### TECHNICAL FIELD

The invention relates to a sensor for providing force feedback, and in particular although not exclusively during surgical procedures. More specifically, one embodiment of the invention relates to a sensor for measuring variations in the stiffness of soft tissue for use in, for example, minimally invasive surgery or catheterisation. Another embodiment relates to a sensor for sensing the force on vasculature or other body channel walls during catheterisation procedures.

### BACKGROUND TO THE INVENTION AND PRIOR ART

Minimally Invasive Surgery

Minimally invasive surgery (MIS) can be described as a form of surgery that is performed through a number of small incisions. The incisions vary in sizes ranging from 3-12 mm in diameter [3]. The incisions are strategically located so as to offer access to the surgical site. A camera is initially inserted through one of the incisions to obtain a field of view of the surgical site. Laparoscopic tools are then inserted through the remaining incisions.

The main advantages of MIS compared to traditional surgery are reduced trauma, postoperative pain, rehabilitation time and improved aesthetics. However, MIS (also referred to as keyhole surgery) does have some major drawbacks according to [4]. First of all, the lack of direct access to the surgical site makes it impossible for surgeons to palpate and feel organs with their hands. In traditional surgery this technique is used to detect any abnormalities that an organ may have such as tumours. Another problem is that the friction between the laparoscopic tools and the trocar port and also the torque required to rotate the tools make it very difficult to sense the actually contact forces between the tool and tissue. This fallback increases the possibility of accidental tissue trauma. The third limitation is the loss of direct hand-eye coordination due to the fact that tools have to move around a fixed point reducing the degrees of freedom to four (excluding the tip's motion): pitch, yaw, roll and insertion [5]. The directions are also reversed in vivo [6] which requires extensive training.

A number of robotic systems have been created to attempt to tackle some of the problems mentioned above in MIS where handheld tools are used. The two most popular systems used for robotic assisted surgery are the Zeus® Surgical System and the da Vinci® Surgical System by Intuitive Surgical Inc. [7] Both Surgical Systems make use of a master-slave design where the master is the surgical console controlled by the surgeon and the slave is the tele-operated cart with a plurality of robotic arms [8]. The main benefits of these systems are the stereoscopic vision which provides a 3 dimensional field of view with depth perception, removal of the fulcrum effect seen in laparoscopy, elimination of tremor, and improved distal dexterity via scaled instrument movements [9]. None of the robotic systems are equipped with high quality feedback; this is of major concern as it can have potentially devastating consequences especially if the tools leave the field of view [5]. The optical camera's field of view can also be obstructed by fluids and other residues during the surgery which makes the need for feedback even more important. There are several types of feedback that need to be distinguished: haptic, tactile and force feedback. Haptics illustrate both cutaneous (tactile) and kinesthetic (force) information, both of these are required to mimic the sensation felt by a human hand [10].

Several sensors have been designed for MIS to overcome the aforementioned problems. The sensing element can be located in four different locations [2] on the laparoscopic tool. The sensor can be placed at or nearby the actuation mechanism. In this location the sensor can measure the stress generated by the mechanical linkages or by the actuator responses. The drawback of this location is that it is far away from the surgical site and therefore the readings can be affected by friction, backlash, inertia and gravity.

The sensor can also be placed on the shaft outside the patient's body. This location benefits from a relative lack of constraints with respect to the size and materials of the sensor as it does not have to go through the incision. The setback with placing the sensor in this location is that it can be affected by the friction and reaction forces from the insertion point.

An alternative location for the sensor is on the shaft inside the patient's body as the friction and reaction forces from the insertion point do not influence the values of the tissue contact forces.

The fourth location of a sensor is at the tip of an instrument that is directly in contact with the organs. This location is advantageous as it is not affected by the other surrounding forces. The drawback with this location is that the space is very limited which generates the need for a very small sensing element. There are several sensing methods that can be used in MIS: displacement-based, current-based, pressure-based, resistive-based, capacity based, piezoelectric-based, vibration based, and optical-based sensing [2].

Recent proposals on MIS-capable rolling indenters for the rapid scanning of the stiffness distribution in soft tissue and the creation of rolling mechanical images show the potential of such methods in a medical environment [1, 11]. The problem associated with the system proposed by [1], which employs a cylindrical wheel attached to a force/torque sensor, is that its manoeuvrability is limited to movements normal to the longitudinal axis of the used cylindrical wheel.

The advantages of providing a surgeon with useful haptic feedback are well documented [2]. Ideally, surgeons would be provided with feedback on the mechanical tissue properties in order to come to a well-informed decision on the location and size of buried tumours and other abnormalities—as is possible through manual palpation in classic surgery. Such feedback would allow surgeons to reduce positive error margins during MIS.

Catheterisation

Another area where haptic feedback is useful for surgeons is in the field of catheterisation. Vascular Interventional Surgery (VIS), or commonly known as catheterisation, is the forwarding of a thin, flexible, small in diameter, elongated tube into the blood vessels, esophagus or urethra. The intent is to reach certain locations and perform examination or offer treatment to tissues and organs within the human body that would otherwise require an open surgery. In cardiac catheterisation the insertion of the catheter can be achieved through a small incision on the groin (upper thigh), the arm, or the neck of the patient, where one of the main blood arteries can be found.

The surgeon has to navigate the catheter through a very delicate and complex network of blood vessels until he/she reaches the required location. Whilst performing the forwarding the surgeon uses visual methods to identify the position of the catheter. The most commonly used visual aids are fluoroscopy, Computed Tomography (CT) and Magnetic Resonance Imaging (MRI).

Fluoroscopy involves the use of an X-ray source and a fluorescent screen to obtain real-time images of the internal structures of a patient, who is placed in-between it. The images are taken in a sequence that can be showed to doctors as a form of movie, representing the internal moving body structures. From this technique two-dimensional images can be acquired, which can either show the skeletal structure (radiographic image), or the vasculature (angiogram) of the patient. In the case of a catheterisation, a contrast agent is injected into the blood vessels to improve their visualisation.

Nonetheless, this technique does come at a cost. The ionising radiation (X-rays), which is emitted by a fluoroscope, generates a health risk for the patient and for the physician performing the examination. This sets constraints to exposure time and the number of radiation doses emitted during the procedure.

Computed Tomography (CT), is another medical imaging technique which is used to allow physicians to obtain two-dimensional images of the interior anatomy of the human body. These images are taken around a single axis of rotation, resulting by some modern CT scanners in the creation of three dimensional images. Therefore, a CT scanner is nothing more than an X-ray machine. The distinction comes with the increased amount of X-ray beams sent to the human body and the varying angles that are propagated. Those distinctive features gives to CT far more detailed imaging results in comparison to a fluoroscope. Having said that, due to the use of ionising radiations (X-rays), physicians do not recommend theses scans without a good reason.

The advantages of non-ionising radiations and their very good imaging capabilities bring MRI scanners to the top of the hierarchy and make them preferable in contrast to other conventional methods used for intervention procedures. An MRI scanner has the ability to examine human soft tissues and networks of blood vessels without the need for a contrast agent, producing three dimensional digital images of superior quality. MRI is capable of not only providing images of the anatomical structures of a human body but it also provide functional information (f-MRI) of them, such as the haemodynamic response related to neural activity in the brain. The physicians have the choice to select the scan plane which is best suited for any examination without the need of repositioning the patient.

However, MRI techniques require the use of tools which do not affect the homogeneity of the magnetic field and its signal to noise ratio, and must be magnetically inert and MRI safe. The uses of components that breach these rules cause either disturbance to the magnetic field and hence artefacts are produced in the resulting images, or make their operation completely useless. Furthermore, the patient's health might be harmed from heating-up effects, or missile effects caused by non-MRI compatible materials which are exposed to the magnetic fields of the scanner.

Apart from the use of visual aids, physicians speak of a haptic feedback which they receive from the interaction of the catheter with the blood vessel. This feedback is used to avoid exerting excessive forces during the catheter's forwarding. Regardless of the haptic sense, physicians are incapable of identifying the origin of the forces. It has been established that there are two different forces that are generated during the navigation of a catheter inside the human body. The first originates from the friction of the catheter's shaft with the blood vessels and the second from the contact of the tip of the catheter with the blood vessel walls.

These two forces are important in a catheterisation, but due to the complexity involved in distinguishing them, only surgeons with vast experience tend to perform them. Therefore, during a manual catheterisation the doctor must rely mostly on the visual guidance offered by one of the above techniques. The force feedback gained from the contact and the friction of the catheter with the blood vessel in most cases stays unexploited.

In the same way as robots developed for general MIS, robots which assist in catheterisation procedures have also been developed. These robots are called

Vascular Interventional Robots (VIR). One of today's most commercial VIR is the Sensei™ robotic catheter system (available from Hansen Medical, Inc., Mountain View, Calif.). This VIR allows the surgeon to perform a catheterization controlling a master-slave robotic system from a distance. In turn, this robot makes use of a modern CT scanner which can produce three dimensional images. This system is also equipped with a force sensor, called Intellisense™ Fine Force Technology, to provide the doctor with a source of force feedback.

Many types of force and pressure sensors have been developed for use in the inside of the vasculature. Those sensors were used for different kinds of measurements, such as blood pressure, oxygen level, blood flow velocity and others. However, very few of them were developed for the detection of contact forces with the blood vessel walls. Such sensors were produced, alone or as part of tactile sensors [14], which employ several sensing principles, such as: strain-gauges [15], piezoresistivity [16]-[18], PVDF films [19] and fibre-optic technology [20]-[23]. The latter principle has been mainly used to measure blood pressure inside the veins.

### SUMMARY OF THE INVENTION

An object of the invention is to provide a sensor which is manoeuvrable and which can be used to give haptic feedback, i.e. tactile and/or force information. In one embodiment this allows the fast creation of mechanical images showing the stiffness and/or force distribution in a soft material and can be used, for example, to identify tissue abnormalities in soft tissue. In another embodiment it allows a catheter tip to be more accurately steered, and particularly by robotic systems, with less risk of tissue damage.

Another object of the invention is to provide a sensor which can be miniaturised so that it can be placed at the tip of an instrument and inserted through small incisions during MIS, or catheterisation.

Embodiments of the invention provide a sensor for detecting the force applied to or by soft material, and that is thus able to measure the stiffness of a soft material. The sensor comprises a sensor body into which is supplied a fluid under pressure. At least one sensor members are provided that are arranged to project from the sensor body under the pressure exerted thereon by the fluid. A sensor member displacement detection system is also provided, that is preferably optically based using optical fibres to illuminate the sensor member and that measures the modulation of the light reflected from the sensor member as the member is displaced against the pressure of the fluid to detect and measure the displacement. From the measured displacement an estimate of the force being applied to the sensor member can be obtained. The sensor is of a small size suitable for use during MIS or catheterisation procedures. Preferably the sensor is constructed of non-metallic material such that it is MRI compatible.

In view of the above from one aspect the invention provides a sensor for measuring a force applied to or by a soft material, the sensor comprising:
a hollow body having a sensor member therein arranged to move relative to the body and, in use, to at least partially project therefrom;
a fluid inlet which, in use, allows a fluid to enter the body under pressure and to exert a pressure against said sensor member so as to cause the at least partial projection of said member; and
a system for measuring displacement of the sensor member against the pressure exerted by said fluid;
wherein the force applied to or by a soft material by or to the sensor member is measured in dependence on the detected displacement of the sensor member.

The advantage provided by this sensor is that, in use, the sensor member effectively “floats” on a cushion of fluid since the sensor member can move relative to the hollow body. This “floating mount” of the sensor member with respect to the sensor body allows the sensor to be used with soft materials that, at least initially, do not present a large reaction force. That is, the “floating mount” provided by the sensor body and the fluid allows the force generated by the sensor pressing against otherwise pliable soft material (or conversely soft material pressing against the sensor) to be sensed in an accurate and reliable manner.

In a preferred embodiment the sensor member is freely moveable with respect to the sensor body. This means that the friction experienced by the sensor member is relatively low so that the sensor can be freely moved across the surface of the soft material under investigation. This allows the sensor to be very manoeuvrable. This overcomes the limitation of the cylindrical wheel approach shown in [1, 11].

Further, the sensor can be manufactured from a relatively small number of components so that it can be made to be very small. This is ideal for MIS, where sensors need to be miniaturised in order to fit through very narrow ports.

The spatial stiffness distribution or the force distribution of a soft material can be measured by the sensor. The fluid exerts a certain pressure on the sensor member and therefore, a certain force. When the sensor is moved over a soft material, the soft material exerts a counterforce on the sensor member which is opposite in direction to the force exerted by the fluid pressure and causes the sensor member to be displaced against the fluid pressure. An area of the soft material which is stiffer than another area will exert a larger force on the sensor member causing it to be displaced by a larger distance against the fluid pressure. Therefore, the degree of displacement of the sensor member is related to the stiffness of the material. By measuring the stiffness of an area of soft material, the sensor can be used to create stiffness distribution maps and/or force maps. These can, for example, aid the identification and classification of abnormal tissue regions.

With regard to the term “stiffness”, an area of the soft material that is “stiffer” than a surrounding area will appear to be harder or less flexible. It will provide greater resistance to the force exerted on it by the sensor member by exerting a greater force on the sensor member compared to a softer area.

The soft material upon which the sensor can be used can be any suitable deformable material. The sensor can be used to determine variations in stiffness on the surface of the soft material as well as at levels at a depth below the surface of the soft material. The soft material can be, for example, soft tissue found in human or animal bodies. Such soft tissue may be internal organs such as the liver or kidney, or may be tubes, ducts or blood vessels. For example, the sensor can be used in urological procedures or in cardiac surgery. Alternatively, the soft tissue may be on the outer body, for example, the skin. For example, the sensor can be used in or to aid breast cancer diagnosis. Alternatively, the sensor can be used for quality inspection of food products in the food industry, for example, the firmness of fruit.

The hollow body can be any suitable hollow body. The hollow body can be made of any suitable material so that it can withstand the pressure of the fluid entering the body. Preferably, the hollow body is made from a material which is bio-compatible and/or can be sterilized. Preferably, the hollow body is made of bio-compatible plastic (e.g. a polymer) or stainless steel. The hollow body could also be made of reinforced epoxy resin. In one embodiment, the hollow body is made from a non-metallic material. Non-metallic materials are more suitable if the sensor is to be used in conjunction with MRI (magnetic resonance imaging) which could be used to guide the sensor to its point of operation. Metallic components cannot be used in an MRI scanner.

Preferably, the hollow body is a tube, and preferably the sensor member is a substantially spherical member. The use of a substantially spherical member has advantages in that the member can then roll across a surface to be measured. In other embodiments, for example where the sensor is being used in a catheter tip, the ability to roll may reduce friction of the sensor as the sensor is moved through a vessel.

Where the sensor member is a substantially spherical member then preferably, the cross-section of the tube has dimensions which are only slightly larger than the diameter of the sensor member. The smallest cross-sectional dimension of the tube (smallest inner diameter if it is a cylindrical tube) may be a few tenths of a millimetre larger than the largest diameter of the substantially spherical member. Preferably, this difference is about 0.01 to 5 mm, more preferably, about 0.01 to 1 mm, and most preferably, about 0.1 to 0.5 mm. Larger gaps can be envisaged, especially when considering larger realisations of such sensor systems which are to be used outside the body or in the food industry. The largest diameter of the sensor member should be between 90% and about 99% of the smallest cross-sectional dimension of the hollow tube. The difference in dimensions will depend in part on the relative sizes of the tube and sensor member. Preferably, the tube is cylindrical. The size of the hollow body can vary in size depending on the application of the sensor. For example, if the sensor is for use in MIS, it may be very small to allow it to be inserted into small incisions in a human body. In preferred embodiments the diameter of the sensor member when substantially spherical may be between about 0.1 mm and about 12 mm, depending on its application. For example, for MIS or external applications, the diameter may be more preferably between about 4 mm and about 8 mm, and most preferably between about 5 mm and about 7 mm. For catheterisation or other internal applications, the diameter may be between 0.1 to 0.8 mm, or more preferably 0.1 to 0.5 mm, or even more preferably, 0.2 to 0.4 mm.

The sensor member may be other shapes, other than substantially spherical. For example, the sensor member may be elliptical, ovoid, or irregularly shaped. Elliptical or ovoid shapes may be preferable in some circumstances as they provide a greater range of movement in terms of the possible displacement against the fluid than is possible with a spherical member. As such, the dynamic range of the sensor may be increased. However, such shapes are not freely rotatable with respect to the sensor body, and hence not as manoueverable. In some embodiments the shape of the sensor member and mounting of the sensor member is such that the sensor member is rollable about at least one axis and more preferably about two or more axes. A substantially spherical member allows rolling of the sensor in any direction, and hence can roll in an infinite number of axes.

Other shapes for the sensor member may also be used. For example, the sensor member may be nail shaped, having a shaft that is able to depress into the sensor body. Preferably the shaft has a head section against which the fluid can act. Preferably the contact end of the sensor member is of a curved profile to reduce friction as the sensor member is passed over a material to be sensed. This is particularly beneficial when the sensor member is of a shape or is mounted such that it cannot roll.

The sensor member can be made from any suitable material. Preferably, the sensor member is made from a material which is bio-compatible and/or can be sterilized. Preferably, the sensor member is made of bio-compatible plastic (e.g. polymer) or stainless steel. In one embodiment, the sensor member is made from a non-metallic material. Non-metallic materials are more suitable if the sensor is to be used in conjunction with MRI (magnetic resonance imaging) which could be used to guide the sensor to its point of operation. Metallic components cannot be used in an MRI scanner. In order for the sensor member to move relative to the hollow body, its diameter should be less than the smallest dimension of the hollow body.

When the hollow body is a cylindrical tube, the sensor member will preferably have a diameter which is only slightly less than the diameter of the tube. This will have the effect of maximising the pressure exerted on the spherical member by the fluid entering the tube. This will also allow the pressure exerted by the fluid on the sensor member to be controlled more easily and, when required, to be kept constant. Preferably, the diameter of the sensor member is about 0.01 to 5 mm less than the diameter of the tube, more preferably, about 0.01 to 1 mm less, more preferably still, about 0.1 to 0.5 mm and, most preferably, about 0.1 to 0.3 mm.

In use, the sensor member partially projects from the hollow body. This allows the sensor member to contact the soft material in order to allow the relative stiffness of the soft material to be measured. Where the sensor member is substantially spherical then it can freely rotate relative to the hollow body, and the sensor can easily be moved over the soft material in any direction. This means the sensor is highly manoeuvrable. Preferably, up to about 50% of the sensor member projects from the hollow body. Preferably, as much of the sensor member as possible should project from the hollow body. This will depend to a certain degree on the way in which the sensor member is retained by the hollow body, and the precise shape of the sensor member. The advantage of a greater projection is that this gives a greater distance through which the sensor member can be displaced when it is caused to move against the pressure exerted by the fluid. This allows the stiffness to be measured more reliably, more accurately and over a wider range. Preferably, between about 10% and about 50% of the diameter of the sensor member projects from the hollow body in a non-displaced state, more preferably, between about 30% and about 50% projects from the hollow body and, most preferably, between about 40% and about 50% projects from the hollow body.

The sensor member can project from the hollow body, for example, through an opening in the hollow body. When the hollow body is a tube, at least one such opening is preferably at one end of the tube. In one embodiment, the smallest diameter of the opening is less than the largest diameter of the sensor member so that the sensor member is retained by the hollow body. In such an embodiment, the size of the opening will affect the distance that the sensor member can project from the hollow body. Preferably, the diameter of the opening is only slightly less than the largest diameter of the sensor member so that as much of the sensor member projects from the hollow body as possible.

In some embodiments, multiple sensor member with corresponding openings in the sensor body can be provided. Preferably the multiple sensor members are distributed around the sensor body so as to provide force measurements in different directions around the sensor body.

The fluid inlet can be any suitable inlet which allows fluid to enter the hollow body under pressure. For example, in one embodiment, the fluid inlet may be an opening to which an air compressor can be connected. There may be a plurality of fluid inlets in the hollow body. The fluid which enters the hollow body can be any suitable fluid which, under pressure, can exert a pressure against the sensor member. Preferably the fluid is a gas, such as air or carbon dioxide. In other embodiments the fluid may be a liquid, such as saline solution. The pressure exerted on the sensor member by the fluid should be constant so that displacement of the sensor member can be related to the stiffness of the soft material. This is because the displacement of the sensor member by a certain force will depend on the pressure, and therefore the force, exerted on the sensor member by the fluid. By keeping the pressure constant, the displacement of the sensor member by a certain force should remain constant. However, different pressures can be used to probe different depths of the soft material. Therefore, the pressure of the fluid may vary from application to application depending on the material being probed and the depth of the layer of material that is to be probed. The greater the pressure, the greater the depth of the soft material that can be probed. During an examination of soft material using the sensor, the distance between the tube and the tissue should be kept constant. Variations in this distance would lead to undesired variations in stiffness/force measurements.

The sensor may be suitable for use inside the human body. For example, in some embodiments the sensor is of a size so as to be insertable into openings created by MIS surgery. In other embodiments the sensor may be incorporated into a catheter tip and/or body. In such a case, the sensor may be used to detect the force applied by the catheter tip or body on the vessel walls through which the catheter is being inserted.

In one embodiment, the sensor further comprises a device for measuring the pressure exerted on the sensor member by the fluid. Any suitable device can be used to measure the pressure and such devices are well known to those skilled in the art. For example, a pressure gauge or barometer could be used.

In another embodiment, the sensor further comprises a device for measuring the flow of fluid entering the hollow body. Any suitable device can be used to measure the fluid flow and such devices are well known to those skilled in the art. For example, an airflow meter could be used.

In yet another embodiment, the sensor may comprise both a device for measuring the pressure exerted on the sensor member by the fluid and a device for measuring the flow of fluid entering the hollow body.

The use of a device for measuring the pressure of the fluid on the sensor member and/or a device for measuring the fluid flow in the hollow body help to determine the force that is exerted on the spherical member by the fluid. This can allow precise measurement of the stiffness of a soft material or the force exerted by or on the soft material rather than just relative values and variations.

The system for detecting displacement of the sensor member against the pressure exerted by the fluid can be any suitable system and can be, for example, an optic or electronic based system.

The detection system can be an electronic based system wherein the surface of the sensor member is an electrical conductor. The detection system comprises an electrical circuit in which the displacement of the sensor member causes a change in a property of the circuit allowing detection of the displacement. This electrical property of the circuit can be, for example, resistance, capacitance or induction. In one embodiment, the surface of the sensor member can form part of an electrical circuit wherein the part of the circuit containing the sensor member changes in distance as the sensor member is displaced. This change in distance alters the electrical properties of that part of the circuit. With suitable calibration, this change can be measured in order to calculate the displacement of the sensor member. The sensor member can be kept a part of the electric circuit through the use of wire brushes.

Alternatively, the detection system can be an optical based system. Many suitable optical based systems for measuring distance and changes therein are known. Preferably, the system comprises an optical emitter and an optical collector wherein, in use, light passing from the emitter to the collector is affected by the sensor member so that a property of the light entering the collector varies depending on the displacement of the sensor member into the body. This property can be, for example, the distance that the light has travelled, the phase of the light or the intensity.

The optical emitter can transmit light from a separate source or can itself be a light source. For example, the optical emitter can be an optical fibre which transmits light from an external light source. Alternatively, the optical emitter can be an LED, a Laser, or another light source.

The optical collector can transmit light to a detector or itself can be a detector. For example, the optical collector can be an optical fibre which transmits light to a detector. Alternatively, the light collector can be a device for detecting variations in the properties of light. For example, this could be a light dependent resistor, a photo detector or a light sensitive transistor which can be used to detect variations in the intensity of light.

One suitable system is a “range finder” type system. In such a system, the light emitted from the optical emitter is reflected from the sensor member and passes into the optical collector, wherein displacement of the sensor member causes the length of the light path to vary. In one embodiment, the optical emitter can simply be a light emitting fibre which can be connected to a light source. The light collector can simply be a light collecting fibre which can be connected to a detector of some kind. Light emitted from the light emitting fibre reflects from the sensor member and is collected by the light collecting fibre. A detector connected to the light collecting fibre can then be used to determine the distance and distance changes associated with the spherical member. For example, the light being emitted from the light emitting fibre can be in pulses and the detector can measure the time that the light pulses take to pass from the light source to the detector. This allows calculation of the distance to the sensor member and any changes in the distance. Alternatively, the detector can be an interferometer in which the phase of the collected light is compared to a reference beam. This again allows the calculation of any changes in the displacement of the spherical member.

In another embodiment, the same optical fibre can be used to both emit light on to the sensor member, and collect light reflected therefrom. An optical coupler is used at the distal end of the optical fibre to couple light from a light source (typically an LED or a Laser) into the optical fibre, and to separate out reflected light, reflected from the sensor member. The use of such a single optical fibre with an appropriate optical coupler is advantageous, as it means that only a single optical fibre need be provided into the sensor body per sensor member, and hence the size and cost of the sensor can be reduced.

The advantage of using light fibres to emit and collect the light is that it means that the light source and detector do not have to be attached to the body of the sensor. This means that there are less components in the sensor itself which will enable the sensor to be as small as possible for use in MIS. This is also advantageous if the sensor is to be used within an MRI scanner where metallic or electronic components can not be employed. In this latter respect, preferably plastic optical fibres are used, as these have increased MRI compatibility.

In an alternative system, light from the emitter is directed at the collector wherein displacement of the sensor member causes less light to enter the collector. This results in the intensity of the light entering the collector being reduced. Preferably, the displacement of the sensor member partially brings it into the light path of the beam from the light emitter and thereby partially blocks the light beam. This results in less light entering the light collector. Accordingly, a detector attached to the light collector could measure the change in light intensity. This could be done, for example, through the use of a light dependent resistor, a photo detector or a light sensitive transistor. With suitable calibration, this would allow the displacement of the sensor member to be measured. In this embodiment, the optical emitter could be a light emitting fibre which, in use, could be connected to a light source and the light collector could be a light collecting fibre which, in use, could be connected to a light sensitive detector. Alternatively, the light emitter itself could be a light source, for example, an LED or a Laser and the light collector could be part of the detection system itself, for example, a light dependent resistor. In this way, the sensor does not require as many external components which would simplify its use and allow it to be miniaturised more easily. This could also allow usage of the sensor system in an MRI scanner.

The invention also provides a sensor array comprising a plurality of sensors as described above. Multiple sensors can be connected together to form an array so that the stiffness of a large area of soft material can be measured in a shorter period of time. The sensors in the array can all be the same or some or all of them can be different. For example they may be different sizes or have different detection systems. Further, they can be positioned at varying heights so that multiple depths of the soft material can be probed in a single pass of the array.

The invention also provides a method of measuring the force applied to or by a soft material, the method comprising:
positioning the sensor of the invention at a selected distance from the soft material; and
moving the sensor over the soft material whilst maintaining the selected distance;
wherein the force applied to or by the soft material is measured in dependence on the displacement of the substantially spherical member.

The method can additionally comprise the step of selecting the pressure exerted on the sensor member by the fluid. This allows different depths of soft material to be probed. The greater the pressure exerted on the substantially spherical member by the fluid, the greater the depth of soft material that will be probed by the sensor.

The method can also additionally comprise the step of selecting the distance of the sensor from the soft material. This can also have a bearing on the depth of the material that is probed. The closer the sensor is to the soft material, the greater the depth of soft material that will be probed by the sensor.

As will be appreciated, a combination of the pressure exerted on the sensor member by the fluid and the distance of the sensor from the soft material will affect the depth at which the soft material is probed.

The distance of the sensor from the soft material and the pressure exerted on the sensor member by the fluid in the sensor can be varied, and the soft material probed a number of times in order to determine variation in the stiffness of the soft material at a number of depths. This can provide a layered map of the stiffness of the soft material. This means that lumps in the soft material can be detected even if they are below the surface of the soft material or buried relatively deeply in the soft material.

### BRIEF DESCRIPTION OF THE DRAWINGS

Further features and advantages of the present invention will become apparent from the following description of embodiments of the invention, presented by way of example only, and by reference to the accompanying drawings, wherein like reference numerals refer to like parts, and wherein:

FIG. 1 is a schematic diagram of the overall layout of a sensor;

FIG. 2 is a picture of a sensor, showing an LED, photosensor and sphere rolled over a silicon block with a nodule;

FIG. 3 shows the voltage variation in a detection system for different, numbers of layers of paper over an object;

FIG. 4 is a picture of a silicone block with 3 triangular nodules;

FIG. 5 shows the dimensions of the nodules in millimetres;

FIG. 6 shows the plots of the 6^{th}, 7^{th} and 8^{th} roll of a sensor over a silicone block with nodules;

FIG. 7 shows a three dimensional plot of the silicone block and its nodules using data from the sensor;

FIG. 8 shows a top view of the silicone block with its nodules using data from the sensor;

FIG. 9 is a schematic diagram of a sensor with an alternative detection system;

FIG. 10 shows an electrical circuit used in a detection system of the sensor;

FIG. 11 is a schematic diagram of a sensor with another alternative detection system.

FIG. 12 is a diagram showing a fourth example sensor according to an embodiment of the invention;

FIG. 13 is a photograph of a sensor in accordance with the fourth example;

FIG. 14 is a schematic diagram of a test rig used to test the sensor of the fourth example;

FIG. 15 is a graph of test results of the fourth example sensor;

FIG. 16 is a photograph of a second test set-up for the fourth example sensor;

FIG. 17 is a photograph of a test sample used to test a sensor according to the fourth example;

FIG. 18 is a second photograph of test nodules used to test the sensor according to the fourth example;

FIGS. 19 and 20 are results obtained from a sensor of the fourth example using the test samples of FIGS. 17 and 18;

FIG. 21 is a cutaway diagram of a sensor according to a fifth example of the invention;

FIG. 22 is a photograph of a sensor according to the fifth example;

FIGS. 23 and 24 are schematic diagrams of the arrangement of the sensor members in the fifth example of the invention;

FIG. 25 is a schematic block diagram illustrating the arrangement of a test and calibration rig for use with a sensor according to the fifth example; and

FIG. 26 shows a series of sensor members that may be used to provide additional embodiments of the invention.

### DESCRIPTION OF THE EMBODIMENTS

Several specific embodiments of the invention will now be described.

### EXAMPLE 1

The sensor is a “sphere on air cushion” sensor which can be used as a stiffness and force sensor in MIS and can be used to measure the spatial stiffness/force distribution of soft tissue and identify tissue abnormalities during MIS. The sensor uses a freely moving sphere that can rotate on the spot and can be moved in any direction along a sample surface. More specifically, the sensor uses a sphere floating on an air-cushion to reduce friction and applies pressurized air onto the sphere to deform the tissue while the sensor is rolled over the surface.

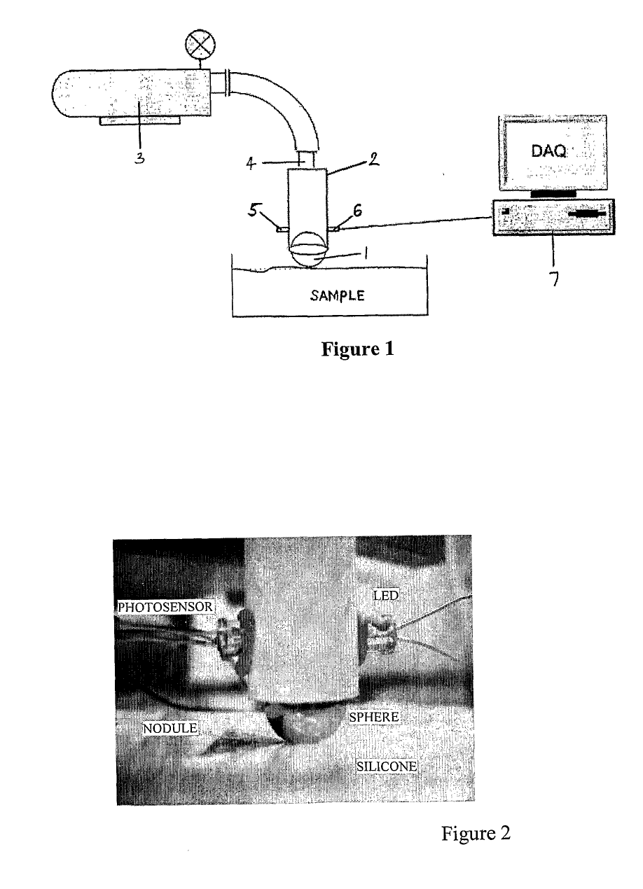

FIG. 1 shows a sensor comprising a 9 mm sphere 1 that is rolled over soft tissue to detect any abnormalities such as lumps. The sphere 1 is situated at the end of a hollow cylindrical shaft 2 with an inner diameter of 11.5 mm and “floats” on a cushion of air generated by an influx of air from a compressor 3 through an inlet 4 at the distal end of the shaft 2. An LED 5 and a photosensitive detector 6 are positioned in the shaft walls opposite each other just above the top of the sphere 1. A beam of light is generated by the LED 5 and is aimed straight at the photosensitive detector 6. The voltage readings generated by the photosensitive detector 6 are amplified by an Operational Amplifier (741) and are then transferred to a data acquisition card in a computer 7. The air pressure generated by the influx of air into the shaft from the compressor 3 is kept constant. A picture of the actual sensor is displayed in FIG. 2.

The sensor system allows the operator to apply forces that are a function of the pressure in the shaft to the probed tissue samples, whilst achieving a virtually friction-free motion of the ball across the tissue surface. Keeping the distance between shaft and tissue constant, any motion of the sphere inside the shaft can be related to the variations of stiffness in the underlying soft material. Applied to spatially distributed stiffness and force sensing in an MIS application, the sensor system can be used to detect stiffness changes such as tumours and other lumps hidden under the surface of a soft tissue organ. As the sphere, for example, is rolled over a lump it is displaced vertically inside the shaft casting a shadow (whose size changes as a function of the sphere's displacement) and thereby reducing the light that is received by the photo sensitive detector. This in turn influences the voltage reading of the photo sensitive detector. Any vertical displacement of the sphere is captured by the detector which allows for the identification of small irregularities and lumps in the soft tissue. The acquired photosensor signals can be used to create a map of the area over which the sphere has been rolled. The force of the sensor can be adjusted by changing the pressure of the air that is exerted onto the sphere. This allows the sensor to be adjusted to probe the tissue at different depths.

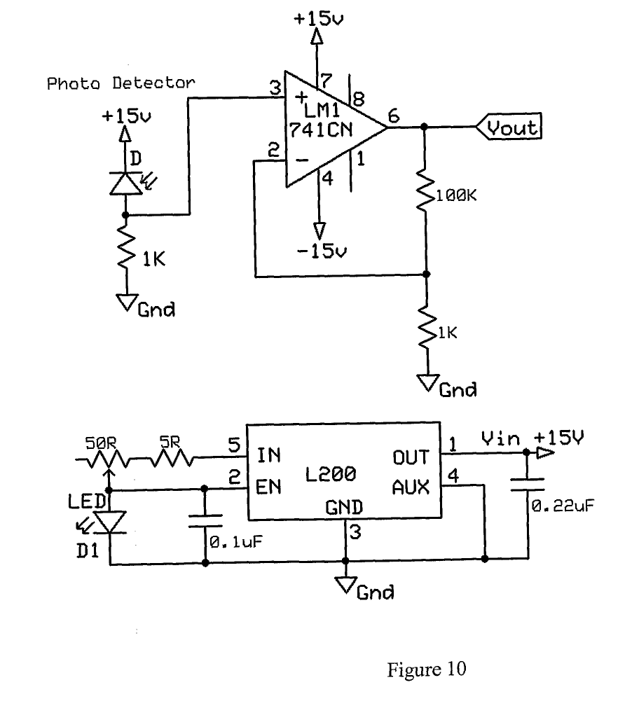

The electrical circuit that powers the sensor consists of an operational amplifier (741), LED (5 mm Superbright), photodiode (5 mm silicon) and 4 resistors of impedance 1.5 kΩ, 10 kΩ, 270 k Ω and 390 kΩ. The circuit is powered by ±15V; a decrease in voltage diminishes the brightness of the LED, consequently also influencing the voltage reading of the photodiode which has a saturation voltage of 10V. The electrical circuit is shown in FIG. 10.

Tactile Testing (Feasibility Study)

The first experiment carried out to assess the effectiveness of the sensor was to determine whether it could detect a subtle irregularity when rolled over a flat surface. The testing surface was made up of a 6 mm diameter paper clipping with a thickness of 103 μm and a number of A4 paper sheets with the same thickness each placed on top of the paper clipping. Experiments were conducted with different numbers of “covering” sheets varying from 1 to 7. The air-cushion sensor was connected to the distal tip of a Mitsubishi RV-6SL 6-DOF robotic manipulator to achieve controlled movements of the sensor in the plane of the paper sheets. The z-axis was normal to the paper sheets (all the sheets of paper used for this experiment were A4, 80 g/m²). The robotic arm was then instructed to roll the sensor over the paper at a constant height to evaluate whether the sensor would detect the “buried” disc of paper. The experiment was repeated by adding an additional sheet of paper on top of the disc until it was covered by 7 layers of paper. Independent tests conducted manually where the researchers probed the test environment with their fingers showed that the clipping was undetectable under 7 layers of paper.

Results

The sensor successfully detected the disc placed under the various layers of papers. The reading given by the sensor when rolled over flat, undisturbed sections of the sheet was 10V. The photosensor was calibrated at a reading of 10V for this vertical position, so that any vertical displacement of the sphere would be detected. The reading when the disc was detected under 3 layers of paper was a value of 9.917V, and under 7 layers of paper a reading of 9.953V was obtained. In order to facilitate the interpretation and plotting of the data, the readings were inverted generating peak values of 0.083 V and 0.047V for the output voltage of the photosensor for 3 and 7 layers respectively.

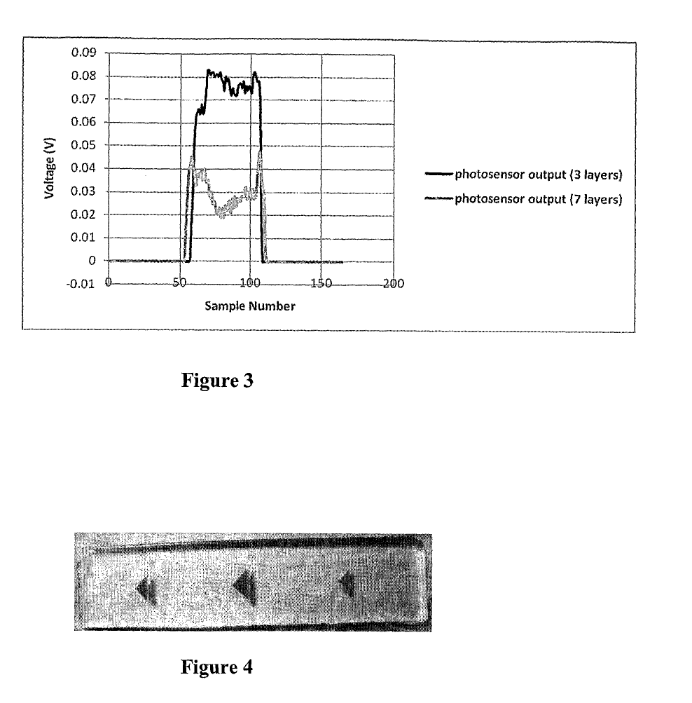

In FIG. 3, the bottom graph shows the trace of the disk under 7 layers of paper and the top graph illustrates the trace under 3 layers of paper. The difference in voltage between the two peaks is 0.036V. The thickness of one layer of A4 (80 g/m²) paper is estimated at 0.103 mm. Four layers generate a thickness of 0.412 mm and a sensitivity of 0.036V. The outcome is a displacement of 0.1 mm leading to a change in voltage of 0.087V. These results are only indicative as the sensor was tested on a non-elastic surface and do not predict the behaviour of the sensor on a soft tissue surface.

Silicone Rollover

For the second experiment, the air-cushion sensor was once again attached to the end effector of the 6DOF robotic manipulator. Data from the photosensitive detector was acquired using a DAQ card and the National Instruments LabView 8.0 software package that is associated with it. The surface on which the sensor was rolled was a silicone rectangloid of length 184 mm, width 60 mm, and height 17 mm illustrated in FIG. 4. In FIG. 4, the triangle on the far left is triangle 1, the centre one is triangle 2 and on the far right is triangle 3.

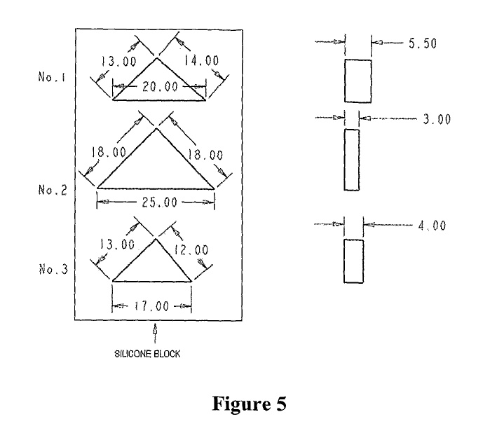

Three nodules of different dimensions (see FIG. 5) are lodged in the silicone block. The nodules are aligned along the central strip of the silicone block with 60 mm separating them.

At the beginning of the experiment, the compressor was set to generate an air pressure of 13895 Pa. The ball (sphere) of the sensor was positioned in the top right hand corner of the silicone block using the robotic manipulator and was rolled to the bottom right hand corner of the block while the readings of the photosensitive detector were recorded for that roll. The sensor was then placed back to the top of the block but shifted to the left by 3 mm from its original position. Another set of data was collected for that second roll. This process was repeated 13 times so that the sensor would have rolled over the entire block in 13 strips, each separated by 3 mm. The distance between the sensor and the silicone block was kept constant at all times. The speed at which the rolls were carried out was also constant.

Results

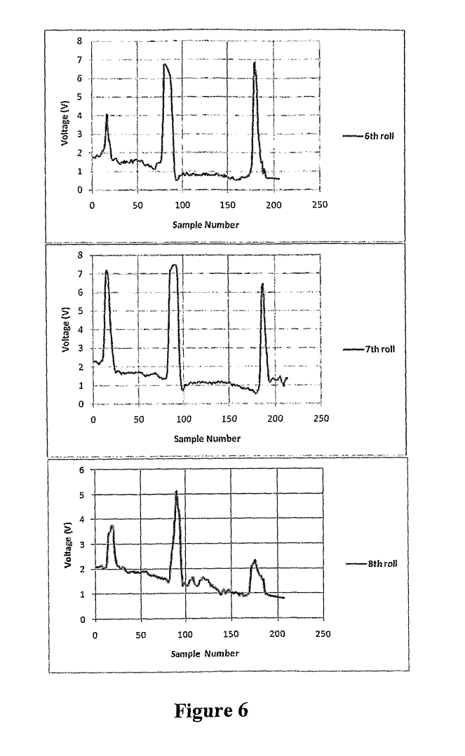

Each one of the 13 vertical rolls of the silicone block generated data from the photosensitive detector. Each strip was plotted to observe whether the nodules were detected by the sensor. Once again, in order to facilitate the interpretation and plotting of the data, the readings were inverted. The plots of the 6th, 7th and 8th roll are illustrated in FIG. 6.

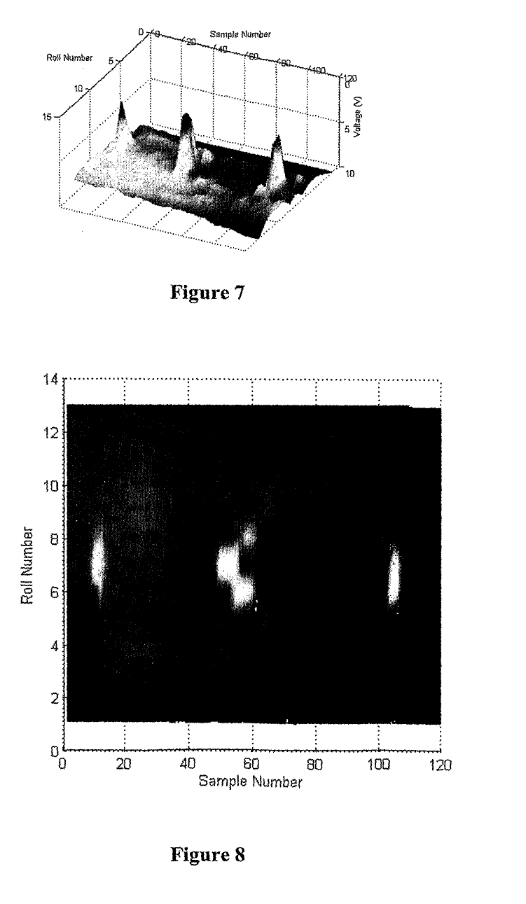

In FIG. 6, the plots visibly illustrate the presence of 3 nodules on each roll. As the plots clearly illustrate the accuracy of the air-cushion sensor, a two dimensional mechanical image (force map) of the silicone block can be generated using the initial readings (see FIG. 7).

The three dimensional representation of the silicone block and the position of its three nodules within the block is clearly portrayed in FIG. 7 illustrating the change in voltage for each roll and sample number. The top view of the silicone in FIG. 8 indicates in white the areas where the nodules are present.

Conclusions

This example presents the feasibility study of an approach of a stiffness/force sensor suitable for measuring mechanical tissue properties during minimally invasive surgery. The sensor makes use of a sphere on an aircushion to interrogate the mechanical properties of soft tissue. Employing a compressor the sphere is pressed against the tissue sample with constant pressure and the generated sphere/tissue interaction can be measured and used to depict the tissue stiffness distribution as well as to detect and locate abnormalities in the scanned tissue. The sensor overcomes the disadvantages of current wheel-based force sensors by improving manoeuvrability allowing unconstrained scanning movements across a tissue's surface. The simplicity of the sensing element (sphere in a shaft) lends itself to straightforward miniaturization with possible applications in areas where access is possible through very narrow ports only. Since all measurements can be conducted optically, there is potential to apply the sensor during operations guided by magnetic resonance imaging, without disturbing or being disrupted by the magnetic fields. The conducted feasibility study shows the potential of this sensor in locating and mapping different levels of tissue stiffness.

### EXAMPLE 2

Alternative Sensor

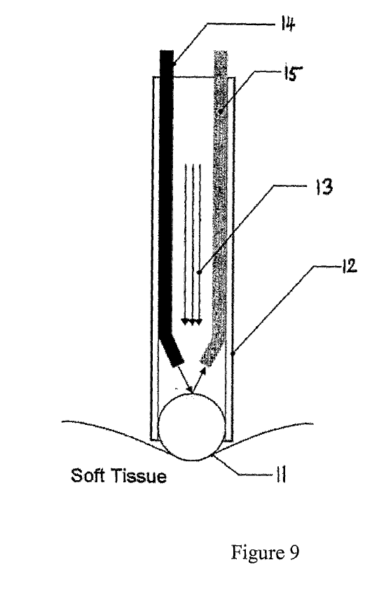

FIG. 9 shows an alternative sensor. The sensor comprises a 9 mm sphere 11 that is rolled over soft tissue to detect any abnormalities such as lumps. The sphere 11 is situated at the end of a hollow cylindrical shaft 12 with an inner diameter of 11.5 mm and “floats” on a cushion of air generated by an influx of air 13 from the distal end of the shaft 12. A light emitting fibre 14 and a light collecting fibre 15 are positioned on the shaft walls opposite each other just above the top of the sphere 11. Light is emitted from the light emitting fibre 14, is reflected off the sphere 11 and is collected by the light collecting fibre 15. The light emitting fibre 14 is connected to a light source, such as an LED or Laser. Light from the light source is transmitted through the light emitting fibre 14 onto the sphere 11. The light collecting fibre 15 is connected to a light sensitive sensor, such as a photo detector, light sensitive transistor or a light sensitive resistor. The intensity of the light reflected off the sphere 11 onto the light collecting fibre 15 is modified by the position of the sphere. As the sphere 11 moves along the shaft 12, the angle of reflection of the light off the sphere 11 is altered hence affecting the readings of the light collecting fibre 15 as it measures the light intensity. This method allows the position of the sphere to be accurately determined.

### EXAMPLE 3

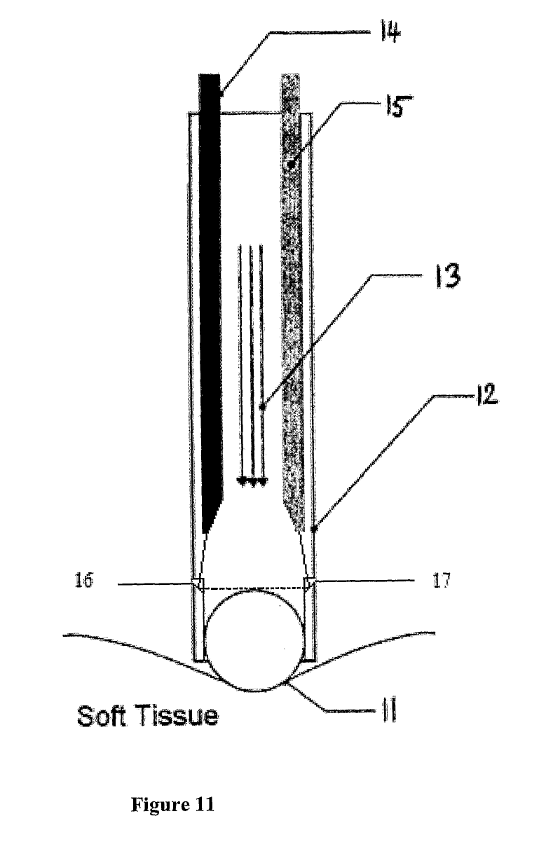

FIG. 11 shows another alternative sensor which is a variation of the sensor of Example 2. The sensor comprises a 9 mm sphere 11 that is rolled over soft tissue to detect any abnormalities such as lumps. The sphere 11 is situated at the end of a hollow cylindrical shaft 12 with a diameter of 11.5 mm and “floats” on a cushion of air generated by an influx of air 13 from the distal end of the shaft 12. A light emitting fibre 14 and a light collecting fibre 15 are positioned on the shaft walls opposite each other both just above a prism 16 and 17 respectively. Light that is shone from the light emitting fibre 14 is reflected off the prism 16 and has a trajectory which is perpendicular to the shaft walls. When the light beam reaches the prism 17 it is reflected straight onto the light collecting fibre thus generating an uninterrupted beam across the cylinder. As the sphere moves along the shaft walls, this beam is partially or fully interrupted which allows the light collecting fibre 15 to perceive a change in light intensity. This change in intensity allows the sensor to accurately know the position of the sphere 11. Prisms 16 and 17 are used so that the sensor is MRI compatible. Instead of prisms small mirrors could be used, if there was no need to achieve MRI compatibility.

### EXAMPLE 4

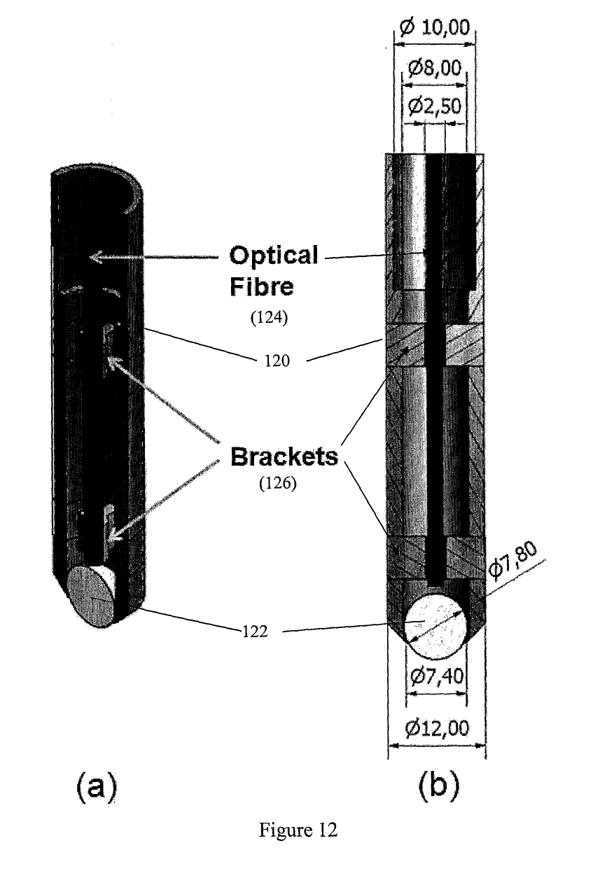

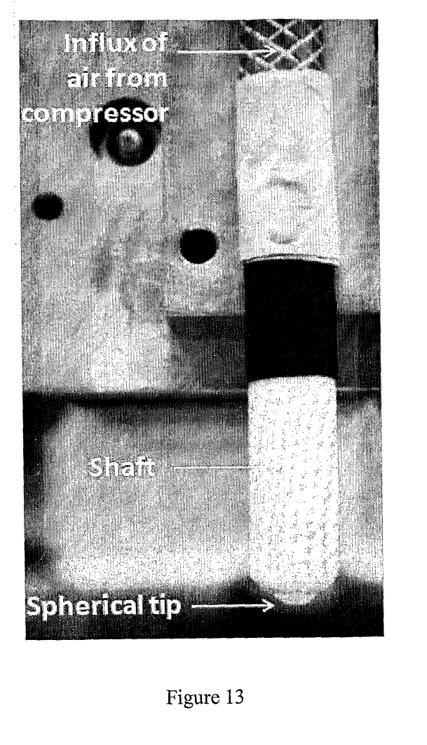

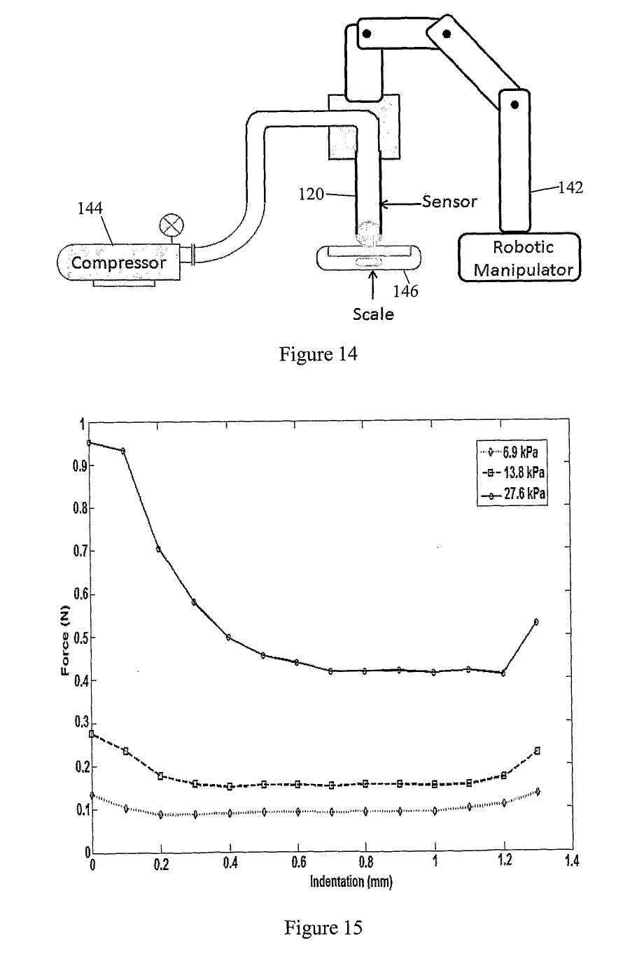

A further, most preferred, example of the invention is shown in FIGS. 12 and 13, and a test rig and test results shown in FIGS. 14 to 20.

More particularly, the proposed sensor 120 is designed to be used as a tactile probe for MIS. The sensor 120 consists of a shaft of outer diameter 12 mm (see FIG. 12b), which is within the acceptable MIS standard. The inner walls of the shaft have a diameter of 8 mm which increases to 10 mm at the distal end and narrows down to 7.4 mm at the tip of the shaft. A 7.8 mm in diameter sphere 122 is located at the tip of the shaft. Two brackets 126 (see FIG. 12a) are placed above each other at distance of 25 mm from one another inside the shaft walls. The brackets have an inner diameter of 2.5 mm and are designed to support an optical fibre 124 that is placed just above the sphere, this is illustrated in FIG. 12a. The optical fibre 124 has an outer jacket of 2.2 mm and a core of 1 mm. The length of the shaft is 60 mm long. The structure of the sensor and the sphere are made out of ABS plastic and were built using the three dimensional rapid prototyping machine Dimension 768. The distal end of the sensor is connected to a compressor and the optical fibre is connected to an electrical circuit.

The motion of the sphere 122 along the longitudinal axis of the shaft is detected by the optical fibre 124. The optical fibre is the one end of a 1×2 optical coupler with 50:50 ratios (Industrial Fiber Optics, Inc., AZ, USA); which is connected to an optical scheme comprising a superbright LED as a light source and a photosensitive detector. The optical fibre 124 inside the shaft both emits and collects the light generated by the optical scheme and reflected off the sphere. As the distance between the tip of the optical fibre and the sphere changes; so does the light intensity collected by the fibre. Initially the sensor is rolled over a flat tissue surface maintaining a distance d1 between the sphere and the tip of the optical fibre and subsequently when it encounter a change in the mechanical properties of the tissue the distance between the tip of the optical fibre 124 and the sphere 122 is reduced to a distance d2. This change in light intensity is reflected by a variation in voltage readings of the optical scheme. The voltage variations are collected using a data acquisition card attached to a computer. The distal end of the probe 120 is connected to the compressor (see FIG. 13) that generates a steady flow of air which applies pressure onto the sphere. As the sphere 122 is rolled over the tissue and pushed along the longitudinal axis of the shaft; it rolls on a cushion of air generating a near frictionless roll. As the distance between the shaft and the tissue under inspection are kept constant; the displacement of the sphere 122 along the longitudinal axis of the shaft indicates a change in the mechanical properties of the surface. This method allows for rapid acquisition of tactile maps of a tissue under inspection.

A number of experiments to characterise the performance of the above described sensor 120 were performed, as described next.

Experiment 1

A. Sensor's Behaviour Under Loading Conditions

The first experiment carried out was to establish the sensor behaviour under loading conditions. The experimental setup consisted of attaching the sensor to the distal tip of a Mitsubishi RV-6SL 6-DOF robotic manipulator. The sensor was positioned onto a scale with only the tip of the sphere touching the scale. The overall schematic of the experimental set-up is displayed in FIG. 14. The scale used for the experiment had a resolution of 0.02 grams. The weight of the sphere with the compressor turned off was observed to be 0.20 grams. For the experiment the compressor was turned on to generate a constant flow of air. During the experiment the sphere was gradually indented (pushed) (0.1 mm at a time) into the shaft by lowering the sensor onto the scale. The readings of the weight generated for each indentation were taken. The initial position of the sphere was taken when it is fully extended at the tip of the probe and its indentation was measured from that point. The maximum indentation achieved was 1.3 mm. This experiment was carried for three different pressure readings given by the compressor. These were 1 psi, 2 psi and 4 psi which are equivalent to 6.9 kPa, 13.8 kPa and 27.6 kPa, respectively.

B. Results

The results obtained were converted into Newtons and were then plotted against the indentation depth which can be seen in FIG. 15. It can be noted that for all three pressures the force of the probe drops as soon as the sphere is indented into the shaft. It can be observed that for the pressures of 6.9 kPa and 13.8 kPA the force remains relatively constant for indentations ranging from 0.3 mm to 1 mm. For the pressure of 27.6 kPa the force stabilises itself from an indentation of 0.7 mm to 1.1 mm. The force exerted by the sphere increases for indentations from 1.2 mm to 1.4 mm regardless of the pressure. The reason for this is that as the sphere is indented into the shaft more air escapes exerting an additional force onto the surface. Despite this change in behaviour for indentations greater of 1.2 mm the force exerted is never greater than the initial force. This has to be noted as it is of great importance. This enables us to know that when an initial force is set (as it is related to the pressure generated by the compressor) it will never be exceeded regardless of the indentation. This prevents the sensor from exerting any force that may damage the tissue as long as the initial force is set according to the tissue's limitations.

Experiment 2

A. Silicone Rollover

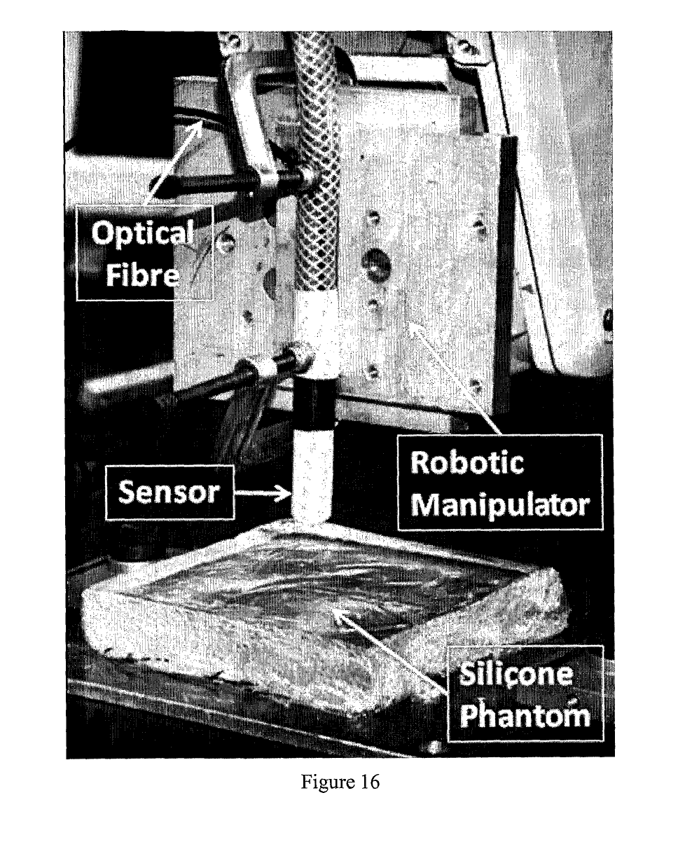

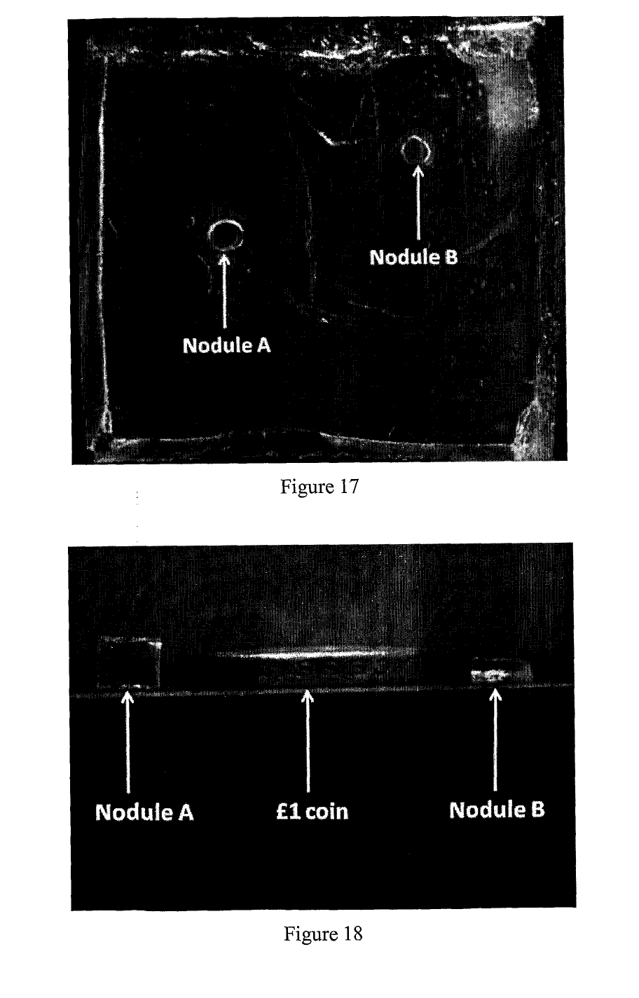

This experiment was carried out to establish the feasibility of this novel air cushion probe as a tactile sensor for MIS. The sensor 120 was attached to the distal tip of the Mitsubishi RV-6SL 6-DOF robotic manipulator to achieve controlled movement over the surface under inspection. Data from the optical scheme was collected using a data acquisition card and the National Instrument LabView 8.0 software package associated with it. The surface that the sensor was rolled over was a silicone phantom of dimensions 100 mm×100 mm and thickness 20 mm. The experimental set up is illustrated in FIG. 16. The silicone phantom does not contain any nodules. Nodules were placed under it which is shown in FIG. 17. The two nodules used were made from a similar silicone mix to the one used for the phantom. This was done to make the task of detecting the nodules more difficult as they had a similar elasticity. This resembles more an in vivo environment where the tumours are also elastic. Nodule A and nodule B were of cylindrical shapes with thicknesses of 6 mm and 2 mm respectively and a diameter of 5 mm. The nodules are illustrated in FIG. 18 next to a one Pound Sterling coin. The nodules were positioned vertically under the silicone phantom just as they are displayed in FIG. 18.

At the start of the experiment the compressor was set to generate a pressure of 6.9 kPa, creating a constant air flow. The sphere was rolled over the silicone block in 8 parallel and adjacent rolls to cover the area under which the nodules were buried. For each roll data was collected based on the readings of the optical fibre which is affected by the motion of the sphere along the longitudinal axis of the shaft. The speed at which the rolls were carried out was kept constant. The indentation depth of the sensor onto the silicone phantom was set to 1.5 mm.

B. Results

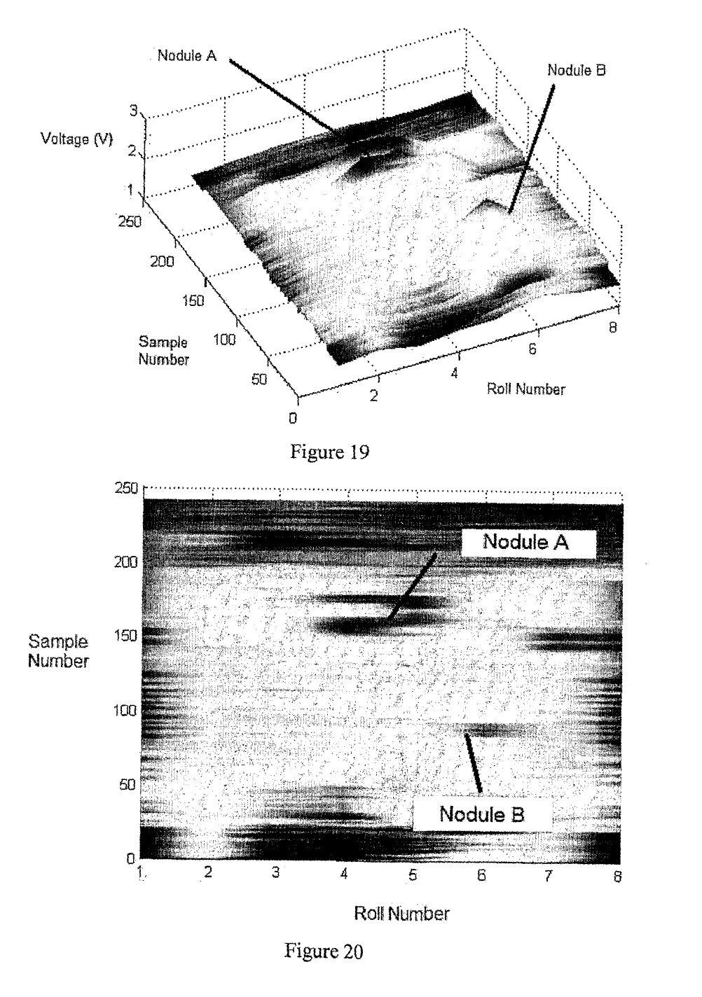

All of the 8 parallel rolls generated data from the optical fibre. This data was used to create a three dimensional map of the silicone block which is illustrated in FIG. 19. The nodule A can clearly be identified where as nodule B is a bit more discreet. The darker areas indicate the highest stiffness area where as the lighter area indicates an area of lesser stiffness. The reason for which the nodule A is surrounded by a vast grey area is that its height of 6 mm raises the surrounding area of the top of the nodules consequently raising the silicone phantom from underneath. The same phenomenon happens with Nodule B but this is less visible as it has a thickness of 2 mm only. A top view of the three dimensional plot is shown in FIG. 20. The two nodules are visible with the top of the nodules shaded dark grey and the surrounding area in lighter grey.

Discussion

The results achieved in both experiments are encouraging. The first experiment illustrated the sensor's behaviour when the sphere is indented into the shaft. This experiment showed how the force exerted by the sensor is altered when the sphere is moved in the longitudinal axis of the shaft and the air flow kept constant. The results of this experiment showed that the force exerted by the sensor is at its maximum when the sphere is fully extended at the tip of the shaft. This finding is reassuring with the knowledge that if the force is set correctly when the sphere is fully extended, no motion of the ball will increase the force of the sensor hence not damaging any tissue under inspection.

The second experiment approximated the behaviour of the sensor when rolled over animal tissue at least to a certain extent. It was impressive to see that nodule B with a thickness of just 2 mm was detected under a silicone phantom of 20 mm. It is even more remarkable as the nodule was made of similar material as the silicone phantom and with properties similar to the animal tissue.

### EXAMPLE 5

A fifth example embodiment will now be described. In the fifth embodiment a sensor specifically designed to aid catheterisation procedures is provided, which makes use of fibre-optic sensors to provide a measure of the force of the catheter tip on the wall of the vessel being used for catheterisation. For example, when vascular catheterisation is being performed, measurements of the force of the catheter tip on the vein or artery walls being traversed can be obtained, thus allowing the risk of tissue damage to be reduced.

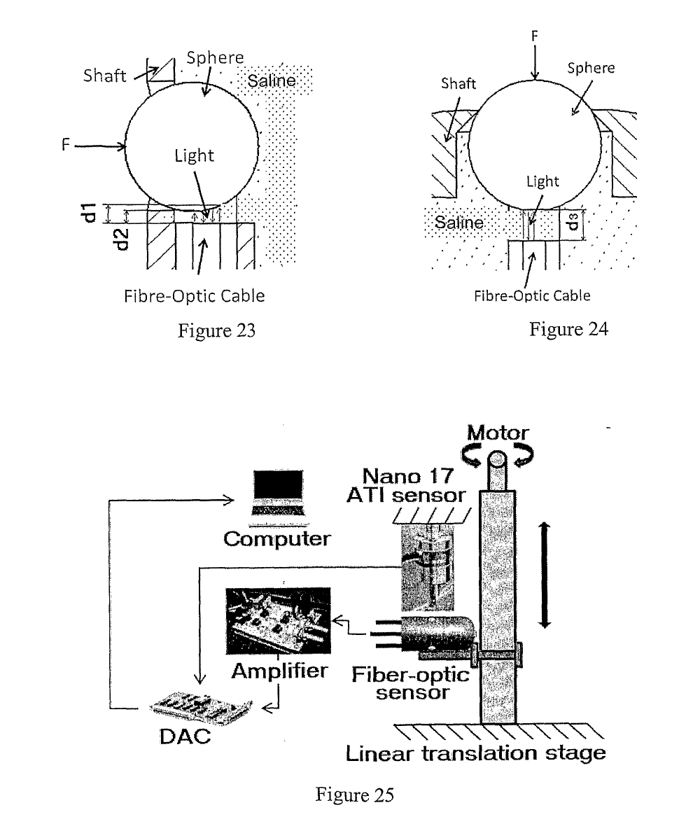

The novel fibre-optic sensor consists of two parts; a cylindrical shaft that serves as the main body of the catheter (A, FIG. 23) and another cylindrical shaft with a curvature at the end of it which represents the tip of the catheter (B, FIG. 23). The entire prototype was made form ABS plastic using a 3D prototyping machine (Dimension 768, Stratasys, Inc.).

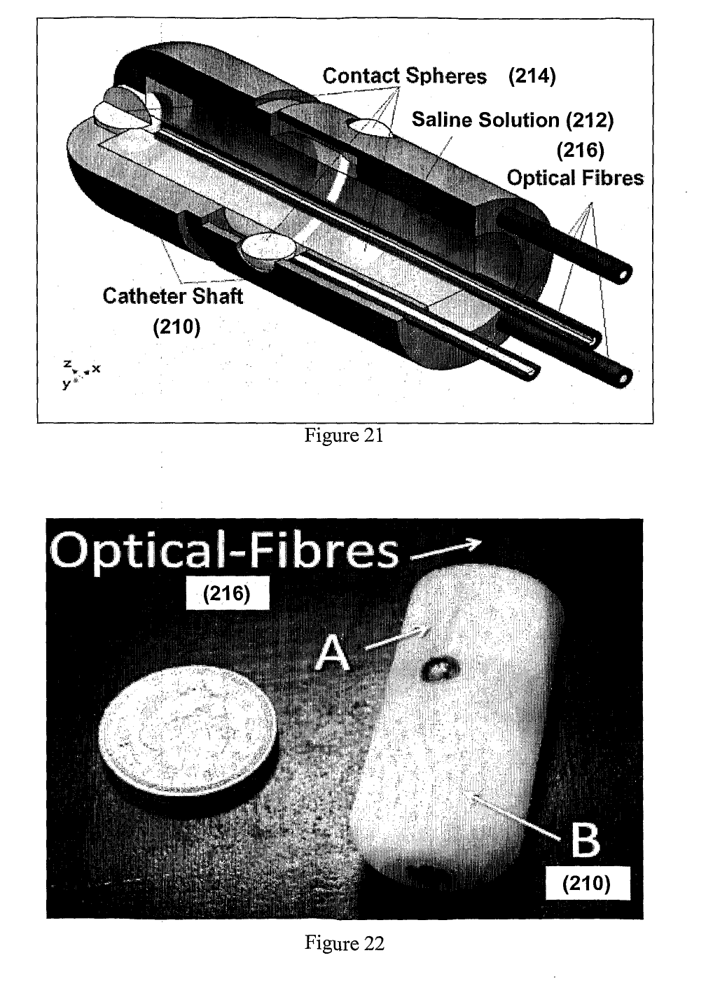

The main body (A) is 25 mm long and hollow. The outer and inner diameters are 20 mm and 12 mm respectively. A gradation at the inner diameter takes place after approximately half way of the shaft's length. The inner diameter is increased to 18 mm in order to allow three peripheral metallic spheres (214, FIG. 21) of 6 mm in diameter to reside in 5 mm in diameter radial openings. The way the spheres are positioned exposes a part of them to the exterior environment. The remaining part of the spheres, which cannot be seen, leaves a 2 mm space from the inner gradation. (See d1 dimension in FIG. 3(a)). At the end of part A, where the optical fibres are shown, three holes of 2.2 mm in diameter are opened in a circular pattern with 120 degrees spacing. These holes are designed to accommodate one 1 mm plastic fibre-optic cable along with their jacket (Edmund Optics Inc., York, UK). These optical-fibres (216, FIG. 21) are inserted up to the point where the ‘step’ is formed by the gradation.

The tip of the prototype sensor (B) is 20 mm long and has the same outer and inner diameter as the main body. These dimensions change after 16 mm of length, where another gradation in the inner of the shaft is formed to create a pocket for the tip sphere. This pocket is 7 mm in diameter and 4 mm in length to allow positioning of a 6 mm sphere.

The sphere 214 is exposing one part to the outer environment through a hole at the top of the shaft. The head of the shaft is given a curved shape to match the appearance of a catheter's tip. The optical-fibres 216 and spheres 214 are placed in position, and the two parts are glued together to form the prototype as seen in FIG. 22. One additional optical-fibre cable is aligned with the inner centre line of the prototype passing through both parts A and B, with its end stopping 2 mm below the inner part of the tip's sphere.

In addition to the optical fibres, the catheter body and tip is also supplied with a saline feed, through a narrow flexible line attached to the end of the catheter body distal from the tip. In use a saline solution is applied at pressure through the line into the cylindrical bodies of the catheter body and tip. The saline solution being under pressure acts against the inner surfaces of the spheres 214, so as to force them outwards of the catheter body and tip against their radial mounts. As mentioned, the radial mounts are 5 mm in diameter, whereas the spheres are 6 mm in diameter, and hence the spheres cannot escape from the catheter body or tip, but instead are projected outwards therefrom by the pressure of the saline solution. However, the arrangements of the radial mounts of the spheres (shown in more detail in FIG. 23 for a side mount in the catheter body, and in FIG. 24 for the tip mount)) are such that the spheres can be pressed into the catheter body or tip against the pressure of the saline solution when the body or tip presses up against a vessel wall through which the catheter is being fed. In this case, saline solution can escape around a sphere when the sphere is pressed into the catheter body or tip. In some embodiments this can be advantageous, as the escaping saline solution can help to lubricate the passage of the catheter. In addition, where the catheter tip is heated and used for ablation of tissue, escape of the saline solution around the spheres can also aid in cooling of the catheter tip and surrounding tissue, once the ablation has taken place.

The saline solution therefore provides a floating mount for the spheres, pressing the spheres outwards of the catheter body, but allowing rotation of the spheres, and for an external force (for example, the reaction force of the vessel wall) to press the spheres inwards against the pressure of the saline solution. This movement of the spheres inwards against the pressure of the saline solution can be detected and measured, and by knowing the saline pressure an estimate of the force being applied by the catheter tip or body to the carrying vessel wall can be obtained. By then knowing this estimate of the force being applied by the catheter, an operator or robot can ensure that tissue damage is avoided.

The basic operating principle of the force detection using the spheres relies on light modulation. An ultra bright red LED emits light at 650 nm wavelengths through the one end of a 1×2 optical coupler with 50:50 ratios (Industrial Fiber Optics, Inc., AZ, USA). The light is guided through a plastic 1 mm optical-fibre to one of the side spheres. There the light travels for a distance of 2 mm (distance d1, FIG. 23) using the fluid as a medium and is reflected back by the sphere's surface. The reflected light travels back using the same optical-fibre and reaches a fibre-optic photodiode detector (SFH 250V, Infineon Technologies Ltd, UK). Therefore, when a lateral force is applied to the sphere, it causes a small displacement of the sphere in the horizontal plane against the pressure of the saline solution. This displacement causes in turn the distance between the sphere and the optical-fibre to decrease (distance d2, FIG. 23). This happens because as the sphere moves laterally the centre line of the optical-fibre aligns with the sphere's South Pole. When the force is removed the pressure of the saline solution moves the sphere back into its initial position. The same light scheme is applied for the remaining two side peripheral spheres.

The tip sphere has a similar operating principle with some differences. The sphere here accepts a force that leads to a vertical displacement against the pressure of the saline solution. This vertical displacement decreases the distance between the South Pole of the sphere and the optical-fibre (distance d3, FIG. 24), changing the light intensity received from the detector.

Similarly, when the load is removed, the pressure of the saline solution returns the sphere to its initial position changing again the intensity of the light.

In all cases the received light signal is converted by the detector into voltage signals in the range of milli-volts. The received voltage signals are amplified using two 741 operational amplifiers in series. On the built circuitry, a low-pass Butterworth filter with cut-off frequency at 10 Hz is used after any amplification, to provide a low signal-to-noise ratio. In addition, dedicated current regulators with the LED's ensure drift-free signals with reduced fluctuations.

To test and calibrate the fibre-optic catheter, a test bench is employed consisting of a) a dc motor (Maxon Motor-118428 with gearhead 275, b) operated linear translational millimetric stage, c) a Nano 17, 6 axes force/torque sensor (ATI Industrial Automation, Inc., NC, USA), d) a NI USB-6211 data acquisition card (National Instruments Corp.), e) a computer with LabView 8.0 and f) a developed amplifying circuit. In FIG. 25 a schematic of the entire test bench is illustrated.

The translational stage is aligned rigidly on a vice to be in a vertical position. It has the ability to move at slow speeds in the vertical axis by the rotating motor attached at the top of its structure with a coupling. A full rotation of the motors shaft corresponds to 1 mm vertical displacement.

In order to test and calibrate the force linearity of the side spheres, the catheter sensor is mounted horizontally on the translational millimetric stage. Saline solution at a known pressure is then supplied to the catheter sensor. The pressure need not be high, just sufficient to project the spheres from the body of the sensor. The dc motor then translates the fibre-optic sensor in the positive and negative vertical directions at a constant speed of 50 μms⁻¹ so as to apply a contact load with the tip of the force/torque sensor (nano 17) directly on the centre of the sphere. The nano 17 is fixed to an external platform which is independent of the test bench surface to reside stationery. Therefore, as the translational stage is bringing the prototype sensor closer to the nano 17, a slowly increasing load is applied to the sphere. The voltage outputs from the electronic circuit amplifier along with the force output from the nano 17 are recorded using the data acquisition card. The rate of acquisition is set at 10 samples/second. The resulting force versus voltage measurements that are obtained thus represent the characteristic of the sensor, for the particular saline solution pressure being applied.

The method to test and calibrate the force linearity of the tip sphere is identical. However, the prototype sensor should be repositioned vertically this time on the translational stage to allow forces to be applied.

Various embodiments of the invention have therefore being described, that provide a sensor for detecting the force applied thereto by the displacement of a sensor member against a fluid pressure. The sensor member is mounted in the sensor body such that the fluid pressure causes the sensor member to project therefrom, whilst preferably allowing free movement of the sensor member. A measurement system that is preferably optically based is used to measure the displacement of the sensor member against the fluid pressure, and thereby, based on the measured displacement, estimate the force being applied to the sensor member by an external body. That force may be a reaction force caused by the sensor being pressed against the external body. In the preferred embodiments the sensor is of a small size suitable for MIS procedures. In another embodiment the sensor is incorporated in a catheter tip.

In the embodiments described above the sensor member that is displaced against the fluid pressure is a substantially spherical member. The use of a substantially spherical member is advantageous, in that it can then roll within its radial mount over the surface being measured. For embodiments such as examples 1 to 4 above, this is clearly advantageous, as the surface being measured can be traversed more easily by the sensor. In addition, for the catheter tip of example 5, the ability of the sensor spheres to roll is also advantageous, in that friction of the catheter tip and body with the vessel wall can be reduced as the catheter is inserted into the vessel.

However, the use of a substantially spherical member can have some disadvantages as well, and in particular in the range of movement of the spherical member, and hence the dynamic range of the sensor. In particular, the range of movement of a spherical sensor member is restricted to that part of the sphere that projects above the radial mount, which is quite small. A greater dynamic range may be obtained with other sensor member shapes.

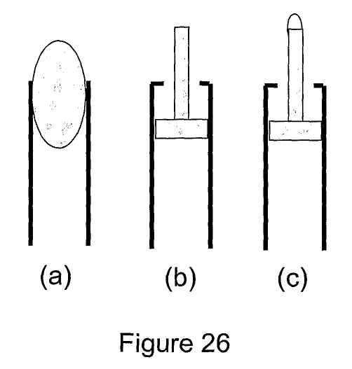

In view of this, the use of a substantially spherical member is not essential, and in other embodiments different shaped sensor members may be used, each of which may have their own shape profiles, which bring advantages in terms of increased dynamic range for the sensor. FIG. 26 shows three example shapes of sensor member, which may be used in different embodiments.

FIG. 26(a) shows that the sensor member may be elliptical in shape. The use of an ellipse is advantageous in that dynamic range of the sensor can be increased, as the range of movement of the ellipse within the radial mount is greater than that of a sphere. However, the use of an ellipse prevents the rolling advantages of a sphere being obtained, although this is somewhat offset by the tip of the sensor being curved, such that a relatively low frictional force is generated as the tip of the elliptical sensor member is moved across a surface.

FIG. 26(b) shows another shape of sensor member. In this case the sensor member is nail shaped, with the nail head retained within the sensor body, and the nail shaft projecting outwards of the sensor body. The radial mount is adapted to allow the nail shaft to project therethrough, but to retain the nail head within the sensor body. Such an arrangement provides the maximum dynamic range for the sensor, as the nail shaft can be made as long as necessary, to give as great a range of movement as necessary. However, the tip of the nail shaft may result in a relatively high frictional force as the sensor tip is moved across a surface.

To alleviate this high frictional force, FIG. 26(c) shows a nail-type sensor member, to provided higher dynamic range, combined with a curved tip to lower the frictional force of the tip as it is moved across the surface. In this case the curved tip is elliptical in shape, although any generally convex curved profile with respect to the nail shaft may be used.

Another modification that may be made to the above described examples is whether the pressurising fluid is released when the sensor member is displaced. In examples 1 to 5 above the use of a spherical sensor member means that the pressurising fluid escapes around the sensor member when the sensor member is displaced into the sensor body by an external force. As discussed, in some embodiments this can be advantageous, in that the escaping pressurising fluid can be used for lubrication and/or cooling purposes. This is particularly advantageous in the field of catheters.

In other embodiments, however, the arrangement may be such that the pressurising fluid is not caused to escape by displacement of the sensor member. For example, with nail-type sensor members such as shown in FIGS. 26(a) and (b), with appropriate seals around the nail-head then displacement of the nail shaft into the sensor body will not necessarily result in escape of the pressurising fluid. In fact, in some such embodiments, the displacement of the sensor into the sensor body will cause the pressure of the pressurising fluid to increase, and this increase in pressure may be monitored (using a pressure sensor) and combined with the displacement detection to provide a more accurate estimate of the force being applied to the sensor member.

Various additional modifications, whether by way of addition, substitution, or deletion, may be made to the above described embodiments to provide further embodiments, any and all of which are intended to be encompassed by the appended claims.

### REFERENCES

1. H. Liu, D. Noonan, K. Althoefer, L. Seneviratne, The Rolling Approach for Soft Tissue modelling and Mechanical Imaging during robot-assisted Minimally Invasive Surgery, to be presented at IEEE ICRA 2008, Pasadena, USA, 19 May 2008.
2. P. Puangmali, K. Althoefer, L. D. Seneviratene, D. Murphy, and P. Dasgupta, State-of-the-Art in Force and Tactile Sensing for Minimally Invasive Surgery, to be published in IEEE Sensors Journal.
3. Bicchi, A., Canepa, G., De Rossi, D., Iacconi, P., & Sci, E. P. (1996). A sensorized minimally invasive surgery tool for detecting tissutal elastic properties. Proc. IEEE Int. Conf. IEEE Robotics and Automation, 884-888.
4. Treat, M. R. (1995). Computer-Integrated Surgery, A Surgeon's Perspective on the Difficulties of Laparoscopic Surgery. MIT Press, 559-560.
5. Tavakoli, M., Aziminejad, R., Patel, R. V., & Moallem, M. (2006). Methods and mechanisms for contact feedback in a robot-assisted minimally invasive environment. Surg Endosc, 20, 1570-1579.
6. Tendick, F., & Cavusoglu, M. C. (1997). Human Machine Interfaces for Minimally Invasive Surgery. Proc. 19th Intl. Conference IEEE/EMBS, 2771-2777.
7. Ballantyne, G. (2002). Robotic surgery, telerobotic surgery, telepresence, and telementoring. Surg Endosc, 20, 1389-1402.
8. D. Noonan, H. Liu, Y. Zweiri, K. Althoefer, L. Seneviratne, A Dual-Function Wheeled Probe for Tissue Viscoelastic Property Identification during Minimally Invasive Surgery, presented at ICRA 2007, pp. 2629-2634, 10-14 Apr. 2007 Roma, Italy.
9. Dario, P., Hannaford, B., & Menciassi, A. (2003). Smart Surgical Tools and Augmenting Devices. IEEE Transaction on Robotics and Automation, 19 (5), 782-792.
10. Okamura, A. M. (2004). Methods for haptic feedback in teleoperated robot-assisted surgery. Industrial Robot: An International Journal, 31 (6), 499-508.
11. P. Puangmali, H. Liu, K. Althoefer, L. Seneviratne, The Optical Fiber Sensor for Soft Tissue Investigation during Minimally Invasive Surgery, to be presented at IEEE ICRA 2008, Pasadena, USA, 19 May 2008
12. Hedrik, A. V., Hisada, M., & Mulloney, B. (2007). Tama-kugel: Hardware and software for measuring direction, distance, and velocity of locomotion by insects. Journal of Neuroscience Methods, 164, 86-92.
13. McGlone, V. A., & Jordan, R. B. (2000). Kiwifruit and apricot firmness measurement by non contact laser air-puff method. Postharvest Biology and Technology, 19, 47-54.
14. Takizawa, H. Tosaka, H. Ohta, R. Kaneko, S. Ueda, Y. “Development of a microfine active bending catheter equipped with MIF tactile sensors.” Twelfth IEEE International Conference on Micro Electro Mechanical Systems. 1999. 412-417.
15. Tanimoto, M., Arai, F., Fukuda, T., Takahashi, I., Negoro, M. “Force Display Method for Intravascular Neurosurgery.” IEEE International Conference on Systems, Man, and Cybernetics. Tokyo, Japan, 1999. 1032-1037.
16. Allen, H., Ramzan, K., Knutti, J., Withers, S. “A Novel Ultra-Miniature Catheter Tip Pressure Sensor Fabricated Using Silicon and Glass Thinning Techniques.” MRS Conference. San Francisco, Calif., 2001. I7.4.
17. Tanase, D., Goosen, J. F. L., Trimp, P. J., French, P. J. “Multi-parameter sensor system with intravascular navigation for catheter/guide wire application.” Sensors and Actuators. Munich, Germany, 2002. 116-124.
18. Tanimoto, M., Arai, F., Fukuda, T., Itoigawa, K., Hashimoto, M., Takahashi, I., Negoro, M. Telesurgery System for Intravascular Neurosurgery. Vol. 1935/2000, in Medical Image Computing and Computer-Assisted Intervention, by MICCAI 2000, CH395. Pittsburgh, Pa., USA: Springer Berlin/Heidelberg, 2000.
19. WeiXing, F., HuanRan, W., ShuXiang, G., KeJun, W., XiuFen, Y. “Design and Experiments of a Catheter Side Wall Tactile Sensor for Minimum Invasive Surgery.” International Conference on Mechatronics and Automation. Harbin, 2007. 1073-1078.
20. Katsumata, T. Haga, Y., Minami, K., Esashi, M. “Micromachined 125 μm diameter ultra miniature fiber-optic pressure sensor for catheter.” Transactions IEE Japan (Transactions IEE Japan) 120, no. 2 (2000): 58-63
21. Strandman, C., Smithb, L., Tenerzb, L., Mk, B. “A production process of silicon sensor elements for a fibre-optic pressure sensor.” Sensors and Actuators 63, no. 1 (1997): 69-74.
22. Cibula, E, Donlagic, D, Stropnik, C. “Miniature fiber optic pressure sensor for medical applications.” Proceedings of IEEE Sensors, 2002, 12-14 Jun. 2002. 711-714.
23. Tohyama, O., Maeda, S., Itoh, H. “Fiber-optic tactile microsensor for detecting the position of the tip of a fiberscope.” Journal of Selected Topics in Quantum Electronics 5, no. 1 (1999): 115-118.

1. A sensor for measuring force applied to or by a soft material, the sensor comprising:
a hollow body having a sensor member therein arranged to move relative to the body and, in use, to at least partially project therefrom;
a fluid inlet which, in use, allows a fluid to enter the body under pressure and to exert a pressure against said sensor member so as to cause the at least partial projection of said member; and
a system for measuring displacement of the sensor member against the pressure exerted by said fluid;
wherein the force applied to or by a soft material is measured in dependence on the detected displacement of the sensor member.

2. The sensor according to claim 1, wherein the hollow body is a tube.

3. The sensor according to claim 2, wherein the tube is substantially cylindrical.

4. The sensor according to claim 2, wherein the sensor member projects from the tube.

5. The sensor according to claim 1, wherein the fluid is a gas.

6. The sensor according to claim 5, wherein the gas is air or carbon dioxide.

7. The sensor according to claim 1, wherein the system is an electronic based system.

8. The sensor according to claim 7, wherein the surface of the sensor member is an electrical conductor and wherein the detection system comprises an electrical circuit in which the displacement of the sensor member causes a change in a property of the circuit allowing detection of the displacement.

9. The sensor according to claim 8, wherein the property of the circuit is resistance, capacitance and or inductance.

10. The sensor according to claim 1, wherein the system is an optical based system.

11. The sensor according to claim 10, wherein the system comprises an optical emitter and an optical collector wherein, in use, light passing from the emitter to the collector is affected by the sensor member so that a property of the light entering the collector varies depending on the displacement of the sensor member.

12. The sensor according to claim 10, wherein the light emitted from the optical emitter is reflected from the spherical member and passes into the optical collector, wherein displacement of the spherical member causes a property of the light to vary.

13. The sensor according to of claim 10, wherein the property of the light that varies is selected from: the distance that the light has travelled; the phase of the light; and the intensity of the light.

14. The sensor according to claim 10, wherein light from the emitter is directed at the collector and wherein displacement of the spherical member causes a different amount of light to enter the collector.

15. The sensor according to claim 14, wherein the displacement of the spherical member at least partially blocks the path of the light from the emitter to the collector causing less light to enter the collector.

16. The sensor according to claim 11, wherein the optical emitter is an optical fibre.

17. The sensor according to claim 11, wherein the optical emitter is an LED or Laser.

18. The sensor according to claim 11, wherein the optical collector is an optical fibre.

19. The sensor according to claim 18, wherein the same optical fibre is used as the optical collector and optical emitter.

20. The sensor according to claim 11, wherein the optical collector is a light dependent resistor, a photo detector or a light sensitive transistor.

21. The sensor according to claim 1, comprising a device for measuring the pressure exerted on the sensor member by the fluid.

22. The sensor according to claim 1, further comprising a device for measuring the flow of fluid entering the hollow body.

23. The sensor of claim 1, wherein the sensor member is a substantially spherical member arranged so as to be freely rotatable with respect to the sensor body.

24. The sensor according to claim 1, and having a plurality of sensor member and displacement detection systems, arranged around the sensor body so as to detect and measure force applied to or by a soft material to or by the sensor in a plurality of directions.

25. The sensor according to claim 1, formed from MRI compatible materials.

26. A sensor array comprising a plurality of sensors according to claim 1.

27. A catheter comprising at least one sensor according to claim 1.

28. A catheter according to claim 27, having a catheter tip and a catheter body, the catheter tip having at least one sensor according to any of the preceding claims, and the catheter body having a plurality of sensors according to claim 1.

29. A method of measuring variations in force applied to or by a soft material, the method comprising:
positioning the sensor according to claim 15 or the sensor array according to claim 26 at a selected distance from the soft material; and
moving the sensor over the soft material whilst maintaining the selected distance;
wherein the force applied to or by the soft material is measured in dependence on the displacement of the sensor member(s).

30. The method of claim 29, additionally comprising the step of selecting the pressure exerted on the sensor member by the fluid.

31. The method of claim 29, additionally comprising the step of selecting the distance of the sensor from the soft material.
